(19) United States
(12) Patent Application Publication
(10) Pub. No.: US 2026/0234109 A1
(43) Pub. Date: Aug. 13, 2026

(54) STABLE ROPIVACAINE CRYSTALS

(71) Applicant: ZHEJIANG CUIZE PHARMACEUTICAL TECHNOLOGY CO., LTD., Hangzhou (CN)

(72) Inventors: Siping SUN, Hangzhou (CN); Qiang GE, Hangzhou (CN); Dingqiang JIANG, Hangzhou (CN); Yi SHEN, Hangzhou (CN); Dingcai LIU, Hangzhou (CN)

(73) Assignee: ZHEJIANG CUIZE PHARMACEUTICAL TECHNOLOGY CO., LTD., Hangzhou (CN)

(21) Appl. No.: 19/278,840

(22) Filed: Jul. 24, 2025

(30) Foreign Application Priority Data
Feb. 11, 2025 (CN) 202510151788.4

(51) Int. Cl. C07D 211/60; A61K 31/445

(52) U.S. Cl. CPC C07D 211/60; A61K 31/445

(57) ABSTRACT

A stable ropivacaine crystal is provided. The ropivacaine crystal has an aspect ratio of approximately less than or equal to 1:(1-2), a particle size distribution width value of less than or equal to 2, a small specific surface area, a stable structure, and the slowest release rate in a dissolution test, thereby achieving better clinical advantages.

### CROSS REFERENCE TO THE RELATED APPLICATIONS

This application is based upon and claims priority to Chinese Patent Application No. 202510151788.4, filed on Feb. 11, 2025, the entire contents of which are incorporated herein by reference.

### TECHNICAL FIELD

The present disclosure relates to the field of medicine, in particular to ropivacaine crystals having different crystal habits.

### BACKGROUND

Ropivacaine, as a long-acting amide local anesthetic of a pure laevo isomer form, causes a reversible blockade for impulse conduction along nerve fibers by blocking the inflow of sodium ions into a nerve fiber cell membrane, thereby achieving dual anesthetic and analgesic effects. In clinical practice, large doses may be used for surgical anesthesia, while small doses produce sensory blockade (analgesia), accompanied with only localized, nonprogressive motor nerve blockade.

Crystals usually exist in multiple forms, and each form of crystals has its specific morphological characteristics, especially those related to the growth environment, conditions, or crystal structures of the crystals. Crystal habits are usually used to describe some habits or characteristics during the growth of crystals, involving the shapes, sizes, symmetry, surface characteristics and the like of the crystals. Ropivacaine crystals are known to have a plurality of forms, i.e., there are a plurality of crystal habits for ropivacaine. Drug forms of different crystal habits have different effects on the production of preparations and final products, which ultimately affect the drug release and efficacy exertion. Therefore, the preparation of ropivacaine crystals that are stable, convenient for preparation and have good release performances has always been the goal pursued by those skilled in the art.

### SUMMARY

One objective of the present disclosure is to provide a ropivacaine crystal having a specific aspect ratio. As used herein, the “aspect ratio” refers to the ratio of the length (L) to the width (W) of the crystal, as calculated by the following formula:

$$aspect ratio = L / W .$$

In one embodiment of the present disclosure, the ropivacaine crystal has an aspect ratio of approximately 1:(1-2).

Further, the ropivacaine crystal has an aspect ratio of approximately 1:(1-1.9).

Further, the ropivacaine crystal has an aspect ratio of approximately 1:(1-1.8).

Further, the ropivacaine crystal has an aspect ratio of approximately 1:(1-1.7).

Further, the ropivacaine crystal has an aspect ratio of approximately 1:(1-1.6).

Further, the ropivacaine crystal has an aspect ratio of approximately 1:(1-1.5).

Further, the ropivacaine crystal has an aspect ratio of approximately 1:(1-1.4).

Further, the ropivacaine crystal has an aspect ratio of approximately 1:(1-1.3).

Further, the ropivacaine crystal has an aspect ratio of approximately 1:(1-1.2).

Further, the ropivacaine crystal has an aspect ratio of approximately 1:(1-1.1).

Further, the ropivacaine crystal has an aspect ratio of approximately 1:1.

In one embodiment of the present disclosure, at least approximately 80%, at least approximately 85%, at least approximately 90%, or at least approximately 95% of the ropivacaine crystals have an aspect ratio of approximately 1:(1-2).

Further, at least approximately 80%, at least approximately 85%, at least approximately 90%, or at least approximately 95% of the ropivacaine crystals have an aspect ratio of approximately 1:(1-1.9).

Further, at least approximately 80%, at least approximately 85%, at least approximately 90%, or at least approximately 95% of the ropivacaine crystals have an aspect ratio of approximately 1:(1-1.8).

Further, at least approximately 80%, at least approximately 85%, at least approximately 90%, or at least approximately 95% of the ropivacaine crystals have an aspect ratio of approximately 1:(1-1.7).

Further, at least approximately 80%, at least approximately 85%, at least approximately 90%, or at least approximately 95% of the ropivacaine crystals have an aspect ratio of approximately 1:(1-1.6).

Further, at least approximately 80%, at least approximately 85%, at least approximately 90%, or at least approximately 95% of the ropivacaine crystals have an aspect ratio of approximately 1:(1-1.5).

Further, at least approximately 80%, at least approximately 85%, at least approximately 90%, or at least approximately 95% of the ropivacaine crystals have an aspect ratio of approximately 1:(1-1.4).

Further, at least approximately 80%, at least approximately 85%, at least approximately 90%, or at least approximately 95% of the ropivacaine crystals have an aspect ratio of approximately 1:(1-1.3).

Further, at least approximately 80%, at least approximately 85%, at least approximately 90%, or at least approximately 95% of the ropivacaine crystals have an aspect ratio of approximately 1:(1-1.2).

Further, at least approximately 80%, at least approximately 85%, at least approximately 90%, or at least approximately 95% of the ropivacaine crystals have an aspect ratio of approximately 1:(1-1.1).

Further, at least approximately 80%, at least approximately 85%, at least approximately 90%, or at least approximately 95% of the ropivacaine crystals have an aspect ratio of approximately 1:1.

Another objective of the present disclosure is to provide a ropivacaine crystal, wherein D₅₀ of the ropivacaine crystal at normal temperature is approximately 5-10 μm. In the present disclosure, D₅₀ refers to a particle size corresponding to a cumulative particle size distribution percentage of a sample reaching 50%, also known as a median particle size or mid-value particle size, which is often used to indicate an average particle size of the crystal.

Further, D₅₀ of the ropivacaine crystal at room temperature is approximately 5-10 μm.

Further, D₅₀ of the ropivacaine crystal at room temperature is approximately 5.0 μm, 5.1 μm, 5.2 μm, 5.3 μm, 5.4 μm, 5.5 μm, 5.6 μm, 5.7 μm, 5.8 μm, 5.9 μm, 6.0 μm, 6.1 μm, 6.2 μm, 6.3μ, 6.4μ, 6.5 μm, 6.6 μm, 6.7μ, 6.8 μm, 6.9 μm, 7.0μ, 7.1μ, 7.2μ, 7.3 μm, 7.4 μm, 7.5 μm, 7.6 μm, 7.7 μm, 7.8 μm, 7.9 μm, 8.0 μm, 8.1 μm, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, 9.9 μm, or 10.0 μm.

Further, D₅₀ of at least approximately 80%, at least approximately 85%, at least approximately 90%, or at least approximately 95% of the ropivacaine crystals at room temperature is approximately 5-10 μm.

Further, D₅₀ of at least approximately 80%, at least approximately 85%, at least approximately 90%, or at least approximately 95% of the ropivacaine crystals at room temperature is approximately 5.0 μm.

Further, D₅₀ of at least approximately 80%, at least approximately 85%, at least approximately 90%, or at least approximately 95% of the ropivacaine crystals at room temperature is approximately 5.1 μm.

Further, D₅₀ of at least approximately 80%, at least approximately 85%, at least approximately 90%, or at least approximately 95% of the ropivacaine crystals at room temperature is approximately 5.2 μm.

Further, D₅₀ of at least approximately 80%, at least approximately 85%, at least approximately 90%, or at least approximately 95% of the ropivacaine crystals at room temperature is approximately 5.3 μm.

Further, D₅₀ of at least approximately 80%, at least approximately 85%, at least approximately 90%, or at least approximately 95% of the ropivacaine crystals at room temperature is approximately 5.4 μm.

Further, D₅₀ of at least approximately 80%, at least approximately 85%, at least approximately 90%, or at least approximately 95% of the ropivacaine crystals at room temperature is approximately 5.5 μm.

Further, D₅₀ of at least approximately 80%, at least approximately 85%, at least approximately 90%, or at least approximately 95% of the ropivacaine crystals at room temperature is approximately 5.6 μm.

Further, D₅₀ of at least approximately 80%, at least approximately 85%, at least approximately 90%, or at least approximately 95% of the ropivacaine crystals at room temperature is approximately 5.7 μm.

Further, D₅₀ of at least approximately 80%, at least approximately 85%, at least approximately 90%, or at least approximately 95% of the ropivacaine crystals at room temperature is approximately 5.8 μm.

Further, D₅₀ of at least approximately 80%, at least approximately 85%, at least approximately 90%, or at least approximately 95% of the ropivacaine crystals at room temperature is approximately 5.9 μm.

Further, D₅₀ of at least approximately 80%, at least approximately 85%, at least approximately 90%, or at least approximately 95% of the ropivacaine crystals at room temperature is approximately 6.0 μm.

Further, D₅₀ of at least approximately 80%, at least approximately 85%, at least approximately 90%, or at least approximately 95% of the ropivacaine crystals at room temperature is approximately 6.1 μm.

Further, D₅₀ of at least approximately 80%, at least approximately 85%, at least approximately 90%, or at least approximately 95% of the ropivacaine crystals at room temperature is approximately 6.2 μm.

Further, D₅₀ of at least approximately 80%, at least approximately 85%, at least approximately 90%, or at least approximately 95% of the ropivacaine crystals at room temperature is approximately 6.3 μm.

Further, D₅₀ of at least approximately 80%, at least approximately 85%, at least approximately 90%, or at least approximately 95% of the ropivacaine crystals at room temperature is approximately 6.4 μm.

Further, D₅₀ of at least approximately 80%, at least approximately 85%, at least approximately 90%, or at least approximately 95% of the ropivacaine crystals at room temperature is approximately 6.5 μm.

Further, D₅₀ of at least approximately 80%, at least approximately 85%, at least approximately 90%, or at least approximately 95% of the ropivacaine crystals at room temperature is approximately 6.6 μm.

Further, D₅₀ of at least approximately 80%, at least approximately 85%, at least approximately 90%, or at least approximately 95% of the ropivacaine crystals at room temperature is approximately 6.7 μm.

Further, D₅₀ of at least approximately 80%, at least approximately 85%, at least approximately 90%, or at least approximately 95% of the ropivacaine crystals at room temperature is approximately 6.8 μm.

Further, D₅₀ of at least approximately 80%, at least approximately 85%, at least approximately 90%, or at least approximately 95% of the ropivacaine crystals at room temperature is approximately 6.9 μm.

Further, D₅₀ of at least approximately 80%, at least approximately 85%, at least approximately 90%, or at least approximately 95% of the ropivacaine crystals at room temperature is approximately 7.0 μm.

Further, D₅₀ of at least approximately 80%, at least approximately 85%, at least approximately 90%, or at least approximately 95% of the ropivacaine crystals at room temperature is approximately 7.1 μm.

Further, D₅₀ of at least approximately 80%, at least approximately 85%, at least approximately 90%, or at least approximately 95% of the ropivacaine crystals at room temperature is approximately 7.2 μm.

Further, D₅₀ of at least approximately 80%, at least approximately 85%, at least approximately 90%, or at least approximately 95% of the ropivacaine crystals at room temperature is approximately 7.3 μm.

Further, D₅₀ of at least approximately 80%, at least approximately 85%, at least approximately 90%, or at least approximately 95% of the ropivacaine crystals at room temperature is approximately 7.4 μm.

Further, D₅₀ of at least approximately 80%, at least approximately 85%, at least approximately 90%, or at least approximately 95% of the ropivacaine crystals at room temperature is approximately 7.5 μm.

Further, D₅₀ of at least approximately 80%, at least approximately 85%, at least approximately 90%, or at least approximately 95% of the ropivacaine crystals at room temperature is approximately 7.6 μm.

Further, D₅₀ of at least approximately 80%, at least approximately 85%, at least approximately 90%, or at least approximately 95% of the ropivacaine crystals at room temperature is approximately 7.7 μm.

Further, D₅₀ of at least approximately 80%, at least approximately 85%, at least approximately 90%, or at least approximately 95% of the ropivacaine crystals at room temperature is approximately 7.8 μm.

Further, D₅₀ of at least approximately 80%, at least approximately 85%, at least approximately 90%, or at least approximately 95% of the ropivacaine crystals at room temperature is approximately 7.9 μm.

Further, D₅₀ of at least approximately 80%, at least approximately 85%, at least approximately 90%, or at least approximately 95% of the ropivacaine crystals at room temperature is approximately 8.0 μm.

Further, D₅₀ of at least approximately 80%, at least approximately 85%, at least approximately 90%, or at least approximately 95% of the ropivacaine crystals at room temperature is approximately 8.1 μm.

Further, D₅₀ of at least approximately 80%, at least approximately 85%, at least approximately 90%, or at least approximately 95% of the ropivacaine crystals at room temperature is approximately 8.2 μm.

Further, D₅₀ of at least approximately 80%, at least approximately 85%, at least approximately 90%, or at least approximately 95% of the ropivacaine crystals at room temperature is approximately 8.3 μm.

Further, D₅₀ of at least approximately 80%, at least approximately 85%, at least approximately 90%, or at least approximately 95% of the ropivacaine crystals at room temperature is approximately 8.4 μm.

Further, D₅₀ of at least approximately 80%, at least approximately 85%, at least approximately 90%, or at least approximately 95% of the ropivacaine crystals at room temperature is approximately 8.5 μm.

Further, D₅₀ of at least approximately 80%, at least approximately 85%, at least approximately 90%, or at least approximately 95% of the ropivacaine crystals at room temperature is approximately 8.6 μm.

Further, D₅₀ of at least approximately 80%, at least approximately 85%, at least approximately 90%, or at least approximately 95% of the ropivacaine crystals at room temperature is approximately 8.7 μm.

Further, D₅₀ of at least approximately 80%, at least approximately 85%, at least approximately 90%, or at least approximately 95% of the ropivacaine crystals at room temperature is approximately 8.8 μm.

Further, D₅₀ of at least approximately 80%, at least approximately 85%, at least approximately 90%, or at least approximately 95% of the ropivacaine crystals at room temperature is approximately 8.9 μm.

Further, D₅₀ of at least approximately 80%, at least approximately 85%, at least approximately 90%, or at least approximately 95% of the ropivacaine crystals at room temperature is approximately 9.0 μm.

Further, D₅₀ of at least approximately 80%, at least approximately 85%, at least approximately 90%, or at least approximately 95% of the ropivacaine crystals at room temperature is approximately 9.1 μm.

Further, D₅₀ of at least approximately 80%, at least approximately 85%, at least approximately 90%, or at least approximately 95% of the ropivacaine crystals at room temperature is approximately 9.2 μm.

Further, D₅₀ of at least approximately 80%, at least approximately 85%, at least approximately 90%, or at least approximately 95% of the ropivacaine crystals at room temperature is approximately 9.3 μm.

Further, D₅₀ of at least approximately 80%, at least approximately 85%, at least approximately 90%, or at least approximately 95% of the ropivacaine crystals at room temperature is approximately 9.4 μm.

Further, D₅₀ of at least approximately 80%, at least approximately 85%, at least approximately 90%, or at least approximately 95% of the ropivacaine crystals at room temperature is approximately 9.5 μm.

Further, D₅₀ of at least approximately 80%, at least approximately 85%, at least approximately 90%, or at least approximately 95% of the ropivacaine crystals at room temperature is approximately 9.6 μm.

Further, D₅₀ of at least approximately 80%, at least approximately 85%, at least approximately 90%, or at least approximately 95% of the ropivacaine crystals at room temperature is approximately 9.7 μm.

Further, D₅₀ of at least approximately 80%, at least approximately 85%, at least approximately 90%, or at least approximately 95% of the ropivacaine crystals at room temperature is approximately 9.8 μm.

Further, D₅₀ of at least approximately 80%, at least approximately 85%, at least approximately 90%, or at least approximately 95% of the ropivacaine crystals at room temperature is approximately 9.9 μm.

Further, D₅₀ of at least approximately 80%, at least approximately 85%, at least approximately 90%, or at least approximately 95% of the ropivacaine crystals at room temperature is approximately 10.0 μm.

Another objective of the present disclosure is to provide a ropivacaine crystal. The ropivacaine crystal has a specific particle size distribution width (SPAN) value. In the present disclosure, the particle size distribution width (SPAN) value is an indicator that characterizes the crystal particle size distribution or crystal size distribution, and is calculated according to a standard deviation and percentile of the particle size distribution. A specific formula is as follows:

$$SPAN = ( {D}_{90} - {D}_{10} ) / {D}_{50} .$$

Wherein D₉₀ is the particle size distribution where 90% of particles are smaller than this value, i.e., the 90th percentile. D₁₀ is the particle size distribution where 10% of particles are smaller than this value, i.e., the 10th percentile. D₅₀ is the particle size distribution where 50% of particles are smaller than this value, i.e., the median particle size.

In one embodiment of the present disclosure, the ropivacaine crystal has a particle size distribution width (SPAN) value of less than or equal to 2.

In one embodiment of the present disclosure, the ropivacaine crystal has a particle size distribution width (SPAN) value of approximately 1-2.

In one embodiment of the present disclosure, the ropivacaine crystal has a particle size distribution width (SPAN) value of approximately 1-1.9.

In one embodiment of the present disclosure, the ropivacaine crystal has a particle size distribution width (SPAN) value of approximately 1-1.8.

In one embodiment of the present disclosure, the ropivacaine crystal has a particle size distribution width (SPAN) value of approximately 1-1.7.

In one embodiment of the present disclosure, the ropivacaine crystal has a particle size distribution width (SPAN) value of approximately 1-1.6.

In one embodiment of the present disclosure, the ropivacaine crystal has a particle size distribution width (SPAN) value of approximately 1-1.5.

In one embodiment of the present disclosure, the ropivacaine crystal has a particle size distribution width (SPAN) value of approximately 1-1.4.

In one embodiment of the present disclosure, the ropivacaine crystal has a particle size distribution width (SPAN) value of approximately 1-1.3.

In one embodiment of the present disclosure, the ropivacaine crystal has a particle size distribution width (SPAN) value of approximately 1-1.2.

In one embodiment of the present disclosure, the ropivacaine crystal has a particle size distribution width (SPAN) value of approximately 1-1.1.

In one embodiment of the present disclosure, the ropivacaine crystal has a particle size distribution width (SPAN) value of approximately 1.

In one embodiment of the present disclosure, the ropivacaine crystal has a particle size distribution width (SPAN) value of approximately 1, 1.1, 1.2, 1.3, 1.4, 1.5, 1.6, 1.7, 1.8, 1.9, or 2.0.

Another objective of the present disclosure is to provide a ropivacaine crystal. The ropivacaine crystal has an XRPD diffraction diagram shown in FIG. 1.

In one embodiment of the present disclosure, the X-ray powder diffraction diagram of the ropivacaine crystal of the present disclosure includes one or more of the following characteristic peaks: 8.894, 10.485, 13.045, 13.580, 14.300, 16.348, 17.918, 19.122, 19.470, 19.984, 20.315, 21.107, 22.567, 23.529, 24.459, 25.593, 26.309, 31.295, 31.880, and 32.449.

In one embodiment of the present disclosure, the ropivacaine crystal of the present disclosure has a particle morphology shown in FIG. 5.

Further, at least approximately 80% of the ropivacaine crystals of the present disclosure have the particle morphology shown in FIG. 5.

Further, at least approximately 85% of the ropivacaine crystals of the present disclosure have the particle morphology shown in FIG. 5.

Further, at least approximately 90% of the ropivacaine crystals of the present disclosure have the particle morphology shown in FIG. 5.

Further, at least approximately 95% of the ropivacaine crystals of the present disclosure have the particle morphology shown in FIG. 5.

The ropivacaine crystal of the present disclosure may be prepared by non-physical methods. The non-physical methods include shearing, cutting, grinding, stamping, etc.

Another objective of the present disclosure is to provide a method for preparing a ropivacaine crystal. The method includes:
dissolving ropivacaine hydrochloride in a solvent, and stirring to fully dissolve;
after complete dissolution, adjusting the pH of the solution to alkalinity and precipitating crystals; and
heating the solution, and performing incubation.

It has been found in the present disclosure that under the premise of not changing crystal lattices, ropivacaine crystals having different crystal habits are obtained when different solvents are selected for crystal precipitation.

In one embodiment of the present disclosure, the solvent is water.

In one embodiment of the present disclosure, when the ropivacaine hydrochloride is dissolved in water, no other auxiliary reagents, such as a dispersant, need to be added.

In one embodiment of the present disclosure, the length-to-width ratio of the obtained ropivacaine crystal is 1:(1-2), the D₅₀ particle size at this time is 5-10 μm, and the particle size distribution width (SPAN) value is less than 2, such that the crystal has the smallest specific surface area, a stable structure, the slowest release rate in a dissolution test, and the strongest sustained-release ability, achieving better clinical advantages.

In one embodiment of the present disclosure, the temperature of crystal precipitation is less than 40° C.

In one embodiment of the present disclosure, the temperature of crystal precipitation is room temperature or normal temperature.

In one embodiment of the present disclosure, adjusting the pH of the solution to alkalinity means adjusting the pH value to be approximately greater than 7, for example: pH 7.5, pH 8.0, pH 8.5, pH 9.0, pH 9.5, pH 10.0, pH 10.5, pH 11.0, pH 11.5, or pH 12.0. The pH value of the solution may be adjusted by using acids, bases, buffers, etc., which are known in the art. Common acids and bases include hydrochloric acid, sulfuric acid, organic acids, sodium hydroxide, potassium hydroxide, phosphate buffers, etc. There is no restriction on the method for adjusting the pH of the solution in the present disclosure, and other suitable acids or bases can also be used in the methods of the present disclosure.

In general, the pH of the ropivacaine solution is adjusted to a range of 7 to 12, preferably to a range of 8 to 11, and preferably, to a range of 9 to 10.

The inventors have found that the higher the heating temperature, the shorter the time it takes to reach the same particle size. In one embodiment of the present disclosure, the heating temperature of the solution is 80-100° C., for example: 80° C., 81° C., 82° C., 83° C., 84° C., 85° C., 86° C., 87° C., 88° C., 89° C., 90° C., 81° C., 92° C., 93° C., 94° C., 95° C., 96° C., 97° C., 98° C., 99° C., or 100° C.

The inventors have found that the particle size of the ropivacaine crystal increases with the extension of the incubation time. In one embodiment of the present disclosure, when the heating temperature is greater than or equal to 90° C., the incubation time is not less than 10 min. Preferably, the incubation time is 10 min-100 min, for example: not less than 15 min, not less than 20 min, not less than 25 min, not less than 30 min, not less than 35 min, not less than 40 min, not less than 45 min, not less than 50 min, not less than 55 min, not less than 60 min, not less than 65 min, not less than 70 min, not less than 75 min, not less than 80 min, not less than 85 min, not less than 90 min, not less than 95 min or not less than 100 min.

In one embodiment of the present disclosure, when the incubation temperature is greater than or equal to 80° C. and less than 90° C., the incubation time is not less than 1 h, preferably, the incubation time is 1 h-5 h, for example: not less than 1.5 h, not less than 2 h, not less than 2.5 h, not less than 3 h, not less than 3.5 h, not less than 4 h, not less than 4.5 h, or not less than 5 h.

Another objective of the present disclosure is to provide a pharmaceutical composition, including the aforementioned ropivacaine crystal. In one embodiment of the present disclosure, the pharmaceutical composition may be a solid preparation, a liquid composition, an emulsion, a suspension, a powder injection, a dry preparation or other forms. The pharmaceutical composition optionally contains at least one other component selected from the following pharmaceutically acceptable components according to the nature of application modes and dosage forms, including but not limited to: a carrier, a diluent, an adjuvant, an excipient, a preservative, a filler, a disintegrant, a wetting agent, an emulsifier, a suspension agent, a sweetener, a flavor correctant, a flavoring agent, an antibacterial agent, an antifungal agent, a lubricant, a dispersant, a temperature-sensitive material, a temperature regulator, an adhesive, a stabilizer, a suspension aid, etc. The pharmaceutical composition may be prepared by many known methods in the art.

Another objective of the present disclosure is to provide a method for the treatment or prevention of postoperative pain, including: administrating the aforementioned pharmaceutical composition containing the ropivacaine crystal to an individual in need. The pharmaceutical composition of the present disclosure may be administered to an individual in need by means of topical or systemic administration. Specifically, it may be administered orally, by injection, transdermally, etc., in a dose-unit preparation containing a conventional non-toxic pharmaceutically acceptable carrier.

The drug or pharmaceutical composition of the present disclosure may be delivered parenterally, i.e., by intravenous (i.v.), intra-cerebroventricular (i.e.v.), subcutaneous (s.c.), intraperitoneal (i.p.), intramuscular (i.m.), subcutaneous (s.d.), or intradermal (i.d.) administration, by direct injection, for example, rapid concentrated injection or continuous infusion. Preparations for injection may be presented in the unit dosage form, for example, in ampules or multiple-dose containers added with preservatives. The composition may take the shape of an excipient, and in the form of a suspension, solution or emulsion in an oily or aqueous carrier, and may include a formulation reagent such as an anti-settling agent, a stabilizer and/or a dispersant. Alternatively, the active ingredient may be reconstituted in the powder form with a suitable carrier (e.g., sterile pyrogen-free water) prior to use.

In the present disclosure, by optimizing the preparation process of the ropivacaine crystal, especially selecting the solvent used in the crystal precipitation process, heating and performing incubation, the ropivacaine crystal has specific morphologies, including but not limited to: a specific length-to-width ratio, a specific particle size, a specific distribution width, a specific particle size range, etc. The inventors have found that, by selecting different solvents for crystal precipitation, on the premise of not changing the crystal lattices, the obtained ropivacaine crystal has the smallest specific surface area, stable structure, good redissolution effect when being made into a freeze-drying preparation, slowest release rate in the dissolution test, and strongest sustained-release ability, thereby achieving better clinical advantages.

### BRIEF DESCRIPTION OF THE DRAWINGS

FIG. 1 shows an XRPD profile of a ropivacaine crystal prepared in Example 1.

FIG. 2 shows a morphology of a ropivacaine crystal prepared in Comparative example 1.

FIG. 3 shows a morphology of a ropivacaine crystal prepared in Comparative example 2.

FIG. 4 shows a morphology of a ropivacaine crystal prepared in Comparative example 3.

FIG. 5 shows a morphology of a ropivacaine crystal prepared in Example 1.

FIG. 6A shows a crystal habit diagram during incubation for 0 min under an incubation condition of 90° C.

FIG. 6B shows a crystal habit diagram during incubation for 30 min under an incubation condition of 90° C.

FIG. 6C shows a crystal habit diagram during incubation for 60 min under an incubation condition of 90° C.

FIG. 6D shows a crystal habit diagram during incubation for 90 min under an incubation condition of 90° C.

FIG. 7 shows a raising tendency of particle size distribution (D₅₀ particle size of single crystal) under an incubation condition of 90° C. over the incubation time.

FIG. 8A shows a crystal habit diagram during incubation for 0 h under an incubation condition of 80° C.

FIG. 8B shows a crystal habit diagram during incubation for 1 h under an incubation condition of 80° C.

FIG. 8D shows a crystal habit diagram during incubation for 3 h under an incubation condition of 80° C.

FIG. 8F shows a crystal habit diagram during incubation for 4.5 h under an incubation condition of 80° C.

FIG. 9 shows a raising tendency of particle size distribution (D₅₀ particle size of single crystal) under an incubation condition of 80° C. over the incubation time.

FIG. 10A shows a morphology of a ropivacaine crystal prepared at a crystal precipitation temperature of 40° C.

FIG. 10B shows a morphology of a ropivacaine crystal prepared at a crystal precipitation temperature of 90° C.

FIG. 11 shows in-vitro release curves of Compositions 1-3 and 5.

FIG. 12 shows plasma concentration-time curves in plasma after intramuscular injection of different samples in rats.

FIG. 13 shows a scanning electron microscopy photograph of ropivacaine medicine liquor after crystal precipitation during incubation at 80° C. for 1 h.

FIG. 14 shows a scanning electron microscopy photograph of ropivacaine medicine liquor after crystal precipitation during incubation at 90° C. for 25 min.

### DETAILED DESCRIPTION OF THE EMBODIMENTS

According to the above content of the present disclosure, as well as the general technical knowledge and customary means in the art, other forms of modifications, replacements or alterations may also be made without departing from the above basic technical ideas of the present disclosure.

### I. Definition

Unless otherwise expressly stated, throughout the description and claims, the terms “include” or its transformations such as “including” or “comprising” is to be construed to include the stated element or component without excluding other elements or other components.

The term “optional” or “optionally” means that an event or situation described later may or may not occur, this description including the occurrence of the event or situation and the non-occurrence of the event or situation.

The term “pharmaceutically acceptable” refers to those compounds, materials, compositions and/or dosage forms that are suitable for use in contact with human and animal tissues within a scope of reasonable medical judgment, without excessively toxic, irritating, allergic reactions, or other problems or complications that are proportionate to a reasonable benefit/risk ratio.

The term “treatment” includes suppressing, alleviating, preventing, or eliminating one or more of the symptoms or side effects associated with a disease, condition, or disorder being treated. The term “effective dose” or “therapeutically effective dose” refers to a dose sufficient to treat, suppress, or alleviate one or more of the symptoms of a disease state being treated or to otherwise provide a desired pharmacological and/or physiological effect. The precise dose will vary based on a number of factors, such as variables on which a subject is dependent (e.g., age or immune system health), a disease or condition, and the treatment administered. The effect of the effective dose may be relative to controls. These controls are known in the art and discussed herein and may be, for example, the condition of the subject before or without the administration of a drug or a drug combination, or a combined effect may be compared with an effect of the administration of only one drug in the case of a drug combination.

The term “pharmaceutical composition” means the composition of the present disclosure or a pharmaceutically acceptable salt, and at least one composition selected from the following pharmaceutically acceptable components according to the nature of application modes and dosage forms, including but not limited to: a carrier, a diluent, an adjuvant, an excipient, a preservative, a filler, a disintegrant, a wetting agent, an emulsifier, a suspension agent, a sweetener, a flavor correctant, a flavoring agent, an antibacterial agent, an antifungal agent, a lubricant, a dispersant, a temperature-sensitive material, a temperature regulator, an adhesive, a stabilizer, a suspension aid, etc.

The term “approximately” represents and covers a specified value and a range greater than and less than that value. In some embodiments, the term “approximately” may represent a variation of ±0.1%, ±0.2%, ±0.5%, ±1%, ±2%, ±3%, ±4%, ±5%, ±6%, ±7%, ±8%, ±9%, or ±10%. In some embodiments, the term “approximately” represents, if appropriate, one standard deviation of the specified value±that value.

The endpoints of the range and any value disclosed herein are not limited to such precise range or value, which should be understood to contain values that are close to those ranges or values. In the case of numeric ranges, one or more new numeric ranges may be obtained by combining the endpoint values of each range, the endpoint values of each range and individual point values, and individual point values, and these numeric ranges shall be deemed to be specifically disclosed herein.

In the present disclosure, the term “room temperature” refers to a temperature in a range of 20° C. to 30° C.

In the present disclosure, the definition of crystal habits is consistent with the definition commonly used in the art.

In the present disclosure, the particle size refers specifically to a particle size detected for a suspension after ultrasonic crushing treatment and dispersion treatment (suspension particles are dispersed into individual crystals under a microscope). If the suspension does not undergo dispersion before direct detection of the particle size, the crystals have varying degrees of agglomeration, and the particle size detected at this time cannot represent the true sizes of the crystals.

In the present disclosure, the D₁₀, D₅₀, and D₉₀ all represent the parameters of a particle size. D₁₀ refers to a particle size corresponding to a cumulative particle size distribution percentage of a sample reaching 10%, and its physical meaning is that the particles each having a particle size less than that particle size account for 10%. D₅₀ refers to a particle size corresponding to a cumulative particle size distribution percentage of a sample reaching 50%, and its physical meaning is that the particles each having a particle size greater than that particle size account for 50% and the particles each having a particle size less than that particle size also account for 50%. D₅₀ is also called a median diameter or a mid-value particle size, which is often used to indicate an average particle size of the crystals. D₉₀ refers to a particle size corresponding to a cumulative particle size distribution percentage of a sample reaching 90%, and its physical meaning is that the particles each having a particle size less than that particle size account for 90%.

In the present disclosure, when used in relation to a form, “essentially pure” means the following compound that has a purity in a specific solid form of ropivacaine that is greater than 90 w-%, including greater than 90, 91, 92, 93, 94, 95, 96, 97, 98 and 99 w-%, and also including 100 w-% by the weight of the compound. The remaining materials contain one or more other forms of compounds, or reaction impurities, or processing impurities resulting from their preparation. For example, the crystalline form of ropivacaine may be considered essentially pure because it has a purity greater than 90 w-% as measured by means known and generally accepted in the art at this time, wherein the remaining materials of less than 10 w-% include one or more other forms of ropivacaine, reaction impurities, or processing impurities.

In the present disclosure, the term “pharmaceutically acceptable excipient” refers to a substance that does not exhibit significant pharmacological activities at a given dose and is added to the pharmaceutical composition in addition to active pharmaceutical ingredients. The excipient may act as a vehicle, a diluent, a releasing agent, a disintegrant, a dissolution modifier, an absorption promoter, a stabilizer or a manufacturing aid, etc. The excipient may include a filler (diluent), an adhesive, a disintegrant, a lubricant, and a flow aid.

In the present disclosure, the term “filler” or “diluent” refers to a substance that is used to dilute an active pharmaceutical ingredient prior to delivery. The diluent and the filler may also be used as stabilizers.

In the present disclosure, the term “adhesive” refers to a substance that binds an active pharmaceutical ingredient and a pharmaceutically acceptable excipient together to maintain cohesive and discrete fractions.

In the present disclosure, the term “disintegrant or disintegrating agent” refers to a substance that, after being added to a solid pharmaceutical composition, promotes the breakdown or disintegration of the active pharmaceutical ingredient after administration and allows it to be released as efficiently as possible so that it dissolves rapidly out.

In the present disclosure, the term “lubricant” refers to a substance that is added to a powder blend to prevent compacted powder blocks from adhering to equipment during a tableting or encapsulation process. They facilitate tablets to be ejected from a mold and can improve the powder flow.

In the present disclosure, the term “flow aid” refers to a substance that is used in tablet and capsule formulations to improve the flow characteristic during tablet compression and to produce an anti-caking effect.

In the present disclosure, the term “aspect ratio” refers to the ratio of the length (longest dimension) to the width (shortest dimension perpendicular to the length) of a crystal particle, unless otherwise specified. The aspect ratio may be determined by measuring the dimensions of the crystal from microscopic images, such as optical microscopy or scanning electron microscopy (SEM) images.

As used herein, the term “habit” refers to the characteristic external shape or morphology of a crystal, which reflects the relative growth rates of different crystal faces under specific conditions. The habit of a crystal may include, but is not limited to, forms such as needle-like, plate-like, prismatic, or block-like shapes. The habit may be observed using techniques such as optical microscopy or scanning electron microscopy (SEM).

### II. Examples

The present disclosure is further elaborated below with reference to examples. The description of specific exemplary embodiments of the present disclosure is for explanatory and illustrative purposes. These descriptions are not intended to limit the present disclosure to a precise form as disclosed, and it is clear that many changes and variations may be made according to the teachings of the description of the present disclosure. The purpose of selecting and describing exemplary embodiments is to explain the particular principles of the present disclosure and their practical applications, so that a person skilled in the art can achieve and utilize various exemplary embodiments of the present disclosure and various choices and variations.

The experimental methods used in the following examples are conventional methods unless otherwise specified.

The materials, reagents, etc. used in the following examples unless otherwise specified, may be obtained commercially.

### Materials and Methods

Ropivacaine crystals prepared in the following examples and comparative examples were detected using a Malvern Static Particle Size, Morphology and Chemical Composition Analyzer (model: MOR2810, manufactured by Malvern Panalytial) and X-ray powder diffraction.

### Sample Treatment:

2 ml of medicine liquor and 6 ml of 0.1% Tween 80 were mixed, put in a vial, mixed well, dispersed with Sonics VCX150 Ultrasonic Crusher, and then added dropwise with a dispersant, followed by measurement at an optical concentration of 5%-10%. Ultrasonic crushing parameters were as follows: the power was 37.5 W, ultrasonic treatment was paused for 5 seconds at every 10 seconds, and the ultrasonic treatment was performed cumulatively for 10 min.

Other detection methods and conditions were determined in accordance with the known general methods in the art and did not exceed the scope of the prior art in the art.

### Comparative Example 1

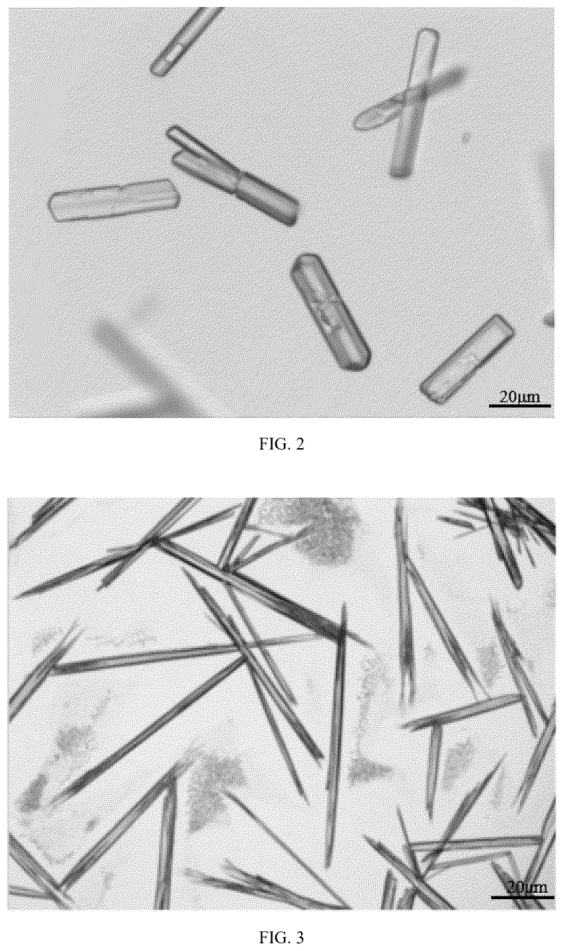

An appropriate amount of ropivacaine was weighed, added with 10 times the weight of ethanol solution, and heated to dissolve at 55° C.; after complete dissolution, the solution was placed at normal temperature for crystal precipitation; and the obtained crystals were separated from the solution. The separated crystals were ultrasonically dispersed according to the sample treatment steps and then observed under a microscope. Results: it was found under the microscope that this crystal had an aspect ratio of 1:(4-10), D₅₀ of 18.6 μm, and SPAN of 3.1, as shown in FIG. 2.

### Comparative Example 2

An appropriate amount of ropivacaine was weighed, added with 10 times the weight of isopropanol solution, and heated to dissolve at 55° C.; after complete dissolution, the solution was placed at normal temperature for crystal precipitation; and the obtained crystals were separated from the solution. The separated crystals were ultrasonically dispersed according to the sample treatment steps and then observed under a microscope. Results: it was found under the microscope that this ropivacaine crystal obtained under this preparation condition had an aspect ratio of greater than 1:10, D₅₀ of 36.4 μm, and SPAN of 5.3, as shown in FIG. 3.

### Comparative Example 3

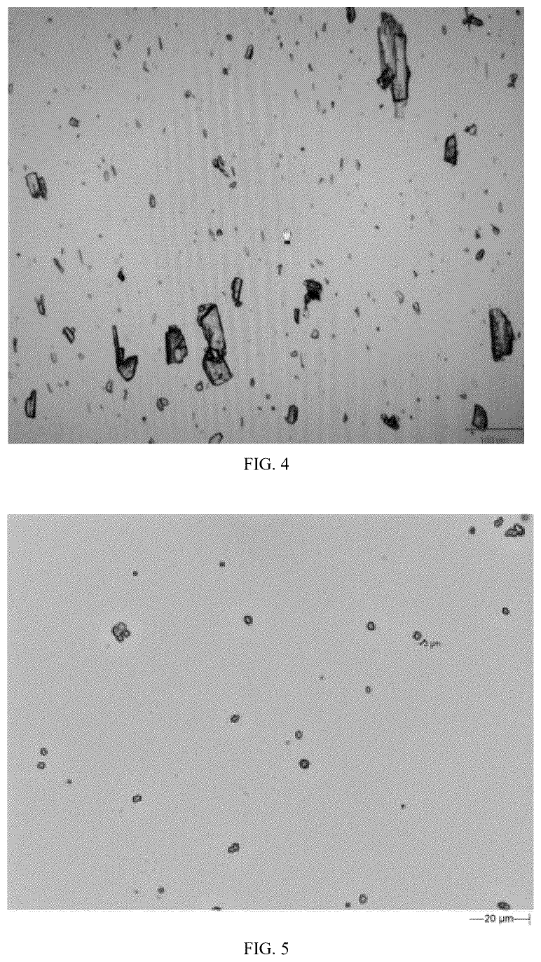

An appropriate amount of ropivacaine was weighed, added into a beaker together with the same weight of freeze-dried protective agent (mannitol), added with an 80% tert-butanol aqueous solution, heated appropriately until stirred to dissolve, and aliquoted into vials (6 ml in each vial), followed by freeze-drying. The freeze-drying parameters were shown in Table 1. The crystal habits of the resulting crystal were detailed in FIG. 4, which was an irregular sheet with large differences in particle size and crystal habit, wherein the length-to-width ratio was difficult to count, and SPAN was 2.8.

TABLE 1
Freeze-drying parameters

| | | | Heating | | Set |
|---|---|---|---|---|---|
| | Set | | (cooling) | | vacuum |
| Freeze-drying phase | temperature | | time | Duration | degree |
| Frozen | −45° | C. | 120 min | 120 min | N/A |
| Primary drying | −30° | C. | 30 min | 300 min | 0.3 mbar |
| phase 1 | | | | | |
| Primary drying | 0° | C. | 30 min | 600 min | 0.3 mbar |
| phase 2 | | | | | |
| Secondary drying | 35° | C. | 60 min | 360 min | 0.02 mbar |
| phase 1 | | | | | |

### Example 1

An appropriate amount of ropivacaine hydrochloride was weighed, added with water, stirred to dissolve at normal temperature, and added with a sodium hydroxide solution to adjust pH >9 after complete dissolution, and the resulting crystal was separated from the solution. The separated crystals were ultrasonically dispersed according to the sample treatment steps and then observed under a microscope. Results: crystal particles prepared by this method were presented as regular cube-like crystal blocks (see FIG. 5), wherein the length-to-width ratio was 1:(1-2), the D₅₀ particle size was 2.55 μm, and the SPAN was 1.6.

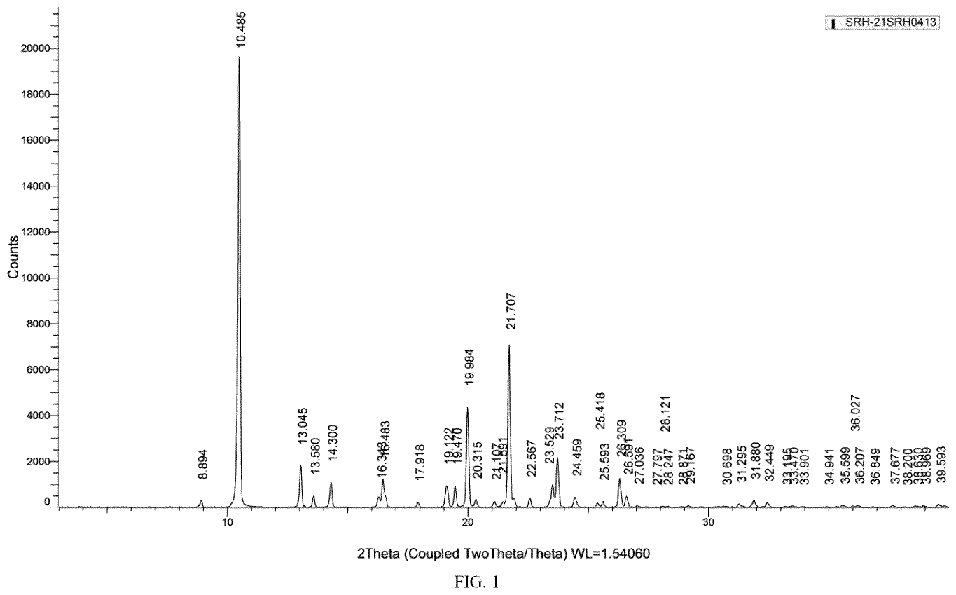

Using an X-ray powder diffraction method, an X-ray powder diffraction profile of the ropivacaine crystal prepared in Example 1 was determined, and the results were shown in FIG. 1. As shown in the XRPD diagram in FIG. 1, the ropivacaine hydrochloride crystal of the present disclosure had the following characteristic peaks: 8.894, 10.485, 13.045, 13.580, 14.300, 16.348, 17.918, 19.122, 19.470, 19.984, 20.315, 21.107, 22.567, 23.529, 24.459, 25.593, 26.309, 31.295, 31.880, 32.449.

### Example 2

In this example, the effects of the incubation temperature and the incubation time on the particle size of the crystal were studied.

An appropriate amount of ropivacaine hydrochloride was weighed, added with water, stirred to dissolve at normal temperature, and added with a sodium hydroxide solution to adjust pH >9 after complete dissolution; and the medicine liquor was then heated to 90° C.-100° C., and subjected to shear dispersion while heating. Samples were taken at different incubation time points to detect changes in particle size and crystal habits of single crystal during heating.

The change tendency of the particle size of the crystal over time under the incubation condition of 90° C. was shown in Table 2. It can be seen from Table 2 that under the incubation condition of 90° C., with the extension of the incubation time, the particle size of single crystal increased, the number of crystals with small particle sizes decreased, the crystal habits and sizes of the crystals tended to be consistent, the particle size distribution width (SPAN) value of the crystal finally decreased to 1.1-1.3 during incubation, and the length-to-width ratio of the crystal was still 1-2.

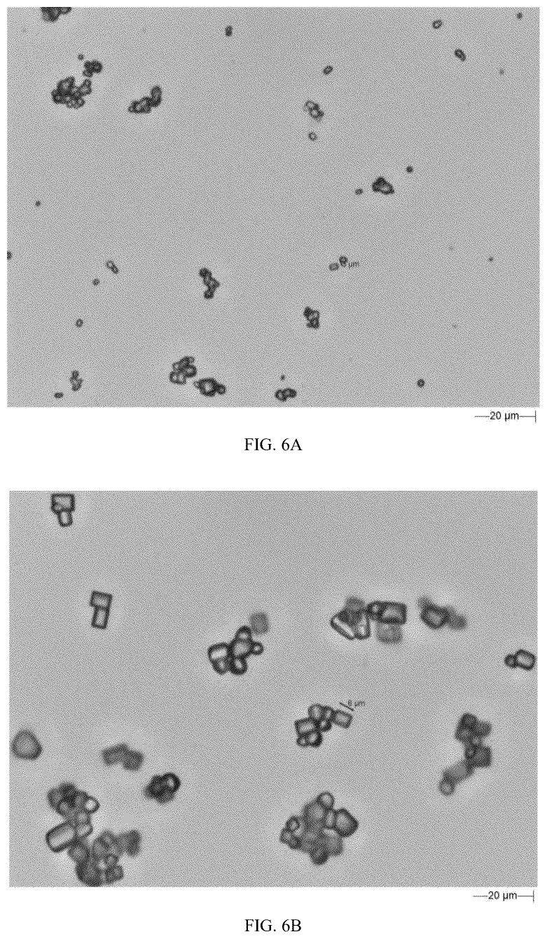

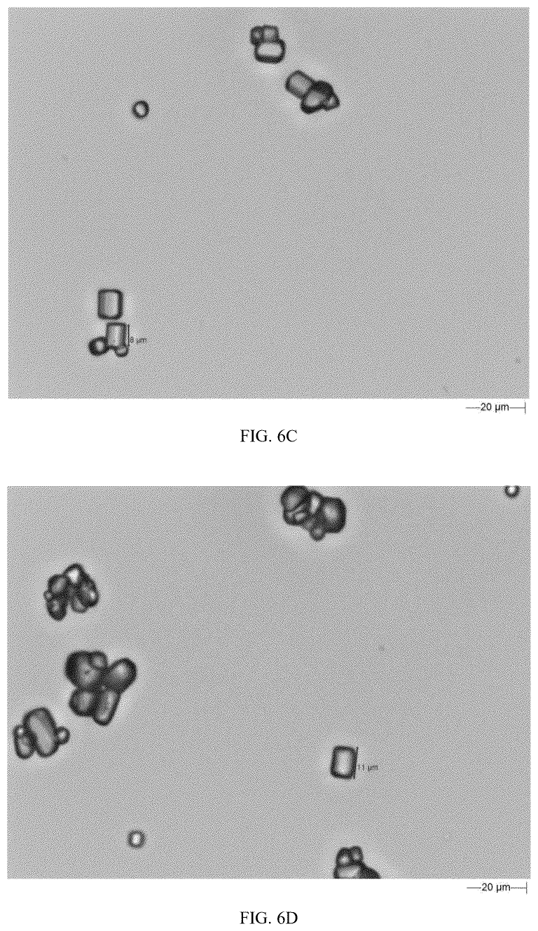

TABLE 2
Change tendency of the particle size
of the crystal over time at 90° C.

| | | | | | Particle size |
|---|---|---|---|---|---|
| | Crystal | D₁₀ | D₅₀ | D₉₀ | distribution |
| Incubation | habit | particle | particle | particle | width |
| time | diagram | size/μm | size/μm | size/μm | (SPAN) |
| 0 min | FIG. 6A | 0.93 | 2.87 | 6.80 | 2.0 |
| 10 min | | 2.47 | 5.71 | 9.81 | 1.3 |
| 20 min | | 2.77 | 5.90 | 9.66 | 1.2 |
| 25 min | | 3.09 | 6.25 | 10.3 | 1.2 |
| 30 min | FIG. 6B | 3.19 | 6.48 | 10.8 | 1.2 |
| 35 min | | 3.19 | 6.59 | 11.1 | 1.2 |
| 40 min | | 3.58 | 6.92 | 11.2 | 1.1 |
| 45 min | | 3.55 | 7.06 | 11.9 | 1.2 |
| 60 min | FIG. 6C | 3.97 | 7.60 | 12.4 | 1.1 |
| 75 min | | 3.71 | 7.40 | 12.4 | 1.2 |
| 90 min | FIG. 6D | 3.98 | 7.81 | 13.4 | 1.2 |

FIGS. 6A-6D, respectively illustrated the crystal habit diagrams of ropivacaine determined at time points of 30 min, 60 min, and 90 min intermediately after the solution was heated to 90° C., respectively. It can be seen from the changes in FIGS. 6A-6D that the particle size of single crystal of ropivacaine increased, the crystal habits were not changed significantly, the crystal habits and sizes of the crystals tended to be the consistent, and the length-to-width ratio of the crystal was still 1-2.

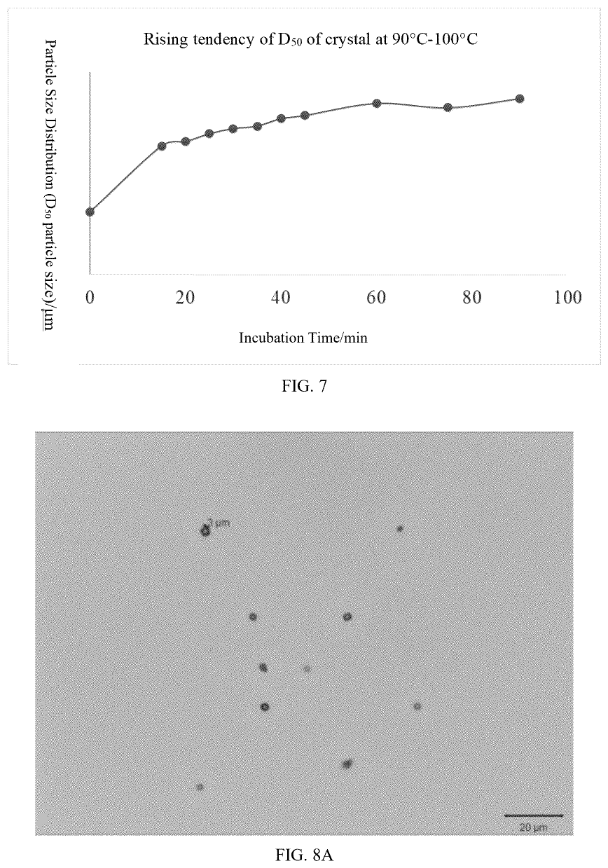

FIG. 7 illustrated, the tendency of the particle size D₅₀ of the ropivacaine crystal over the incubation time under the incubation condition of 90° C. As can be seen from the drawings, the particle size of single crystal increased with the extension of the incubation time.

If the incubation temperature is lowered to 80° C., the growth rate of the crystal decreased, and the tendency of the particle size of the crystal over time was shown in Table 3.

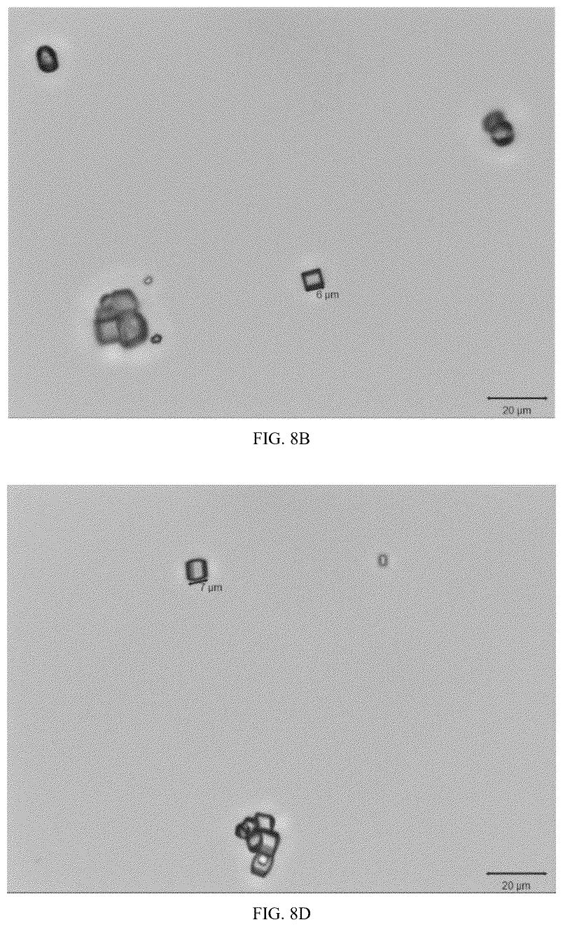

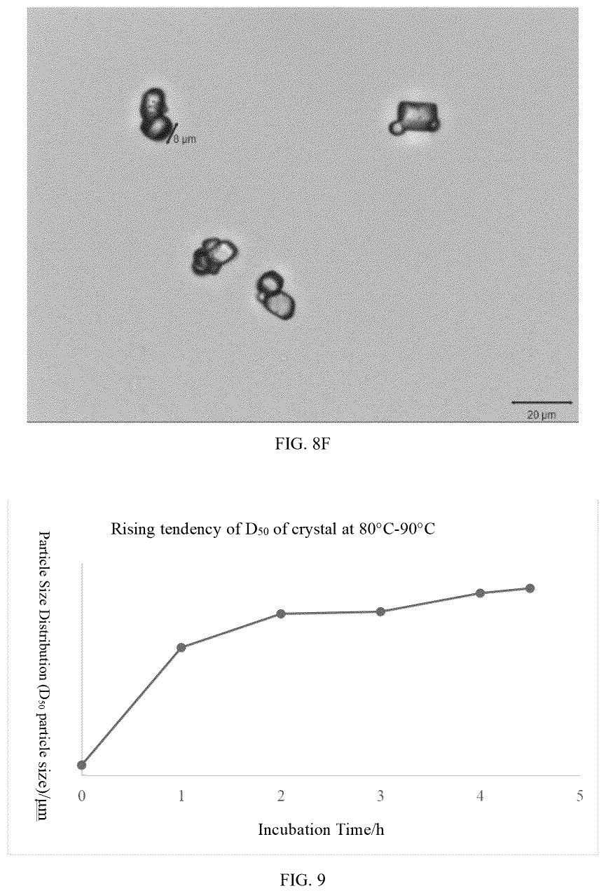

TABLE 3
Change tendency of the particle size
of the crystal over time at 80° C.

| | | | | Particle size | |
|---|---|---|---|---|---|
| | D₁₀ | D₅₀ | D₉₀ | distribution | Crystal |
| Incubation | particle | particle | particle | width | habit |
| time | size/μm | size/μm | size/μm | (SPAN) | diagram |
| 0 h | 0.84 | 2.32 | 5.15 | 1.9 | FIG. 8A |
| 1 h | 1.82 | 6.19 | 10.18 | 1.4 | FIG. 8B |
| 2 h | 2.18 | 7.3 | 11.36 | 1.3 | |
| 3 h | 2.16 | 7.37 | 11.51 | 1.3 | FIG. 8D |
| 4 h | 2.43 | 7.98 | 12.4 | 1.2 | |
| 4.5 h | 2.53 | 8.14 | 12.82 | 1.3 | FIG. 8F |

FIG. 8A, FIG. 8B, FIG. 8D, and FIG. 8F respectively illustrated, the result diagrams of the determination of crystal habits of ropivacaine at different time points after the solution was heated to 80° C., respectively. It can be seen from the changes in FIG. 8A, FIG. 8B, FIG. 8D, and FIG. 8F that with the extension of the incubation time, the particle size of single crystal of ropivacaine increased, the crystal habits and sizes of the crystals tended to be the consistent, the crystal habits were not changed significantly, and the length-to-width ratio of the crystal was still 1-2.

FIG. 9 illustrated, the tendency of the particle size D₅₀ of the ropivacaine crystal over the incubation time under the incubation condition of 80° C. As can be seen from the drawings, the particle size of single crystal increased with the extension of the incubation time, and the rising tendency was close to FIG. 7.

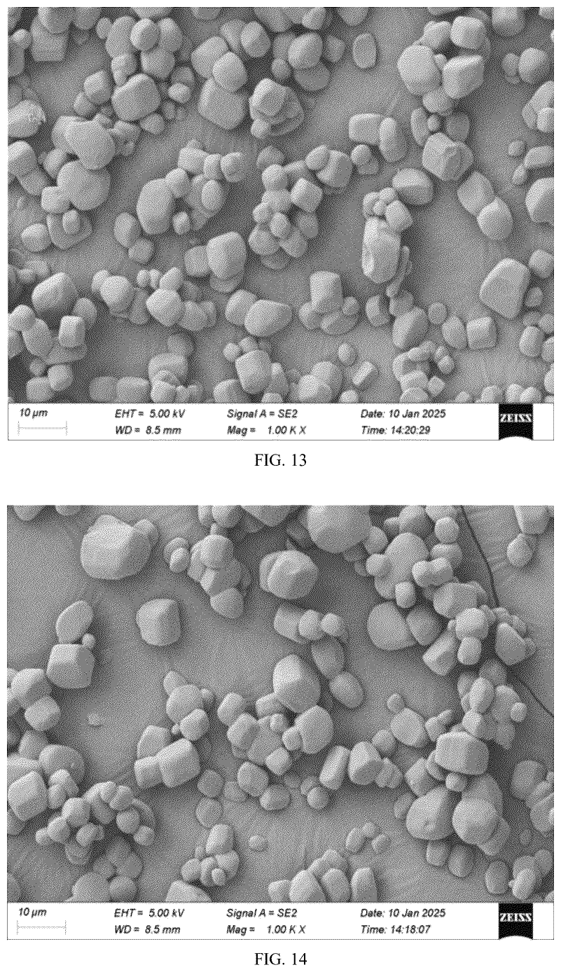

FIG. 13 and FIG. 14 respectively illustrated, scanning electron microscopy photographs of ropivacaine medicine liquor after crystal precipitation during incubation at 80° C. for 1 h and 90° C. for 25 min, respectively, in which incubated crystals had similar crystal habits and particle sizes. Therefore, the incubation process can control the growth of the ropivacaine crystals to a target particle size by increasing the heating temperature and extending the incubation time, without changing the crystal shape, thereby achieving more uniform distribution of the crystals.

### Comparative Example 4

In this comparative example, the effect of the crystal precipitation temperature on the formation of crystal habits was studied.

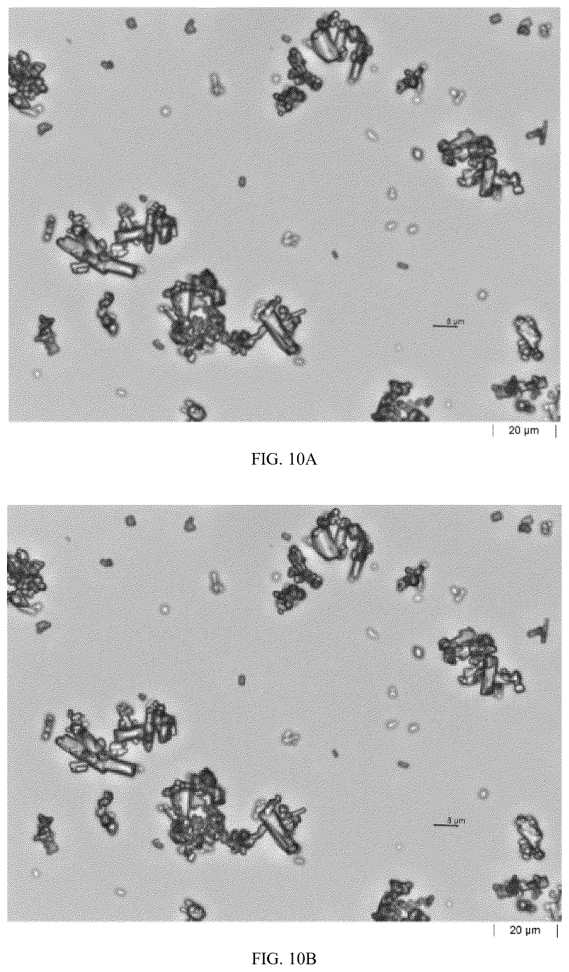

An appropriate amount of ropivacaine hydrochloride was weighed, added with water, stirred to dissolve at 40° C., and added with a sodium hydroxide solution to adjust pH >9, and the resulting crystals were separated from the solution. The separated crystals were ultrasonically dispersed according to the sample treatment steps and then observed under a microscope, as shown in FIG. 10A.

The water temperature of crystal precipitation was changed to 90° C., the experiment was performed repeatedly, the crystals were observed under the microscope, and the results were shown in FIG. 10B.

As can be seen from FIGS. 10A-10B, the ropivacaine crystals at 40° C.-90° C. were both rod-shaped and blocky, indicating that the crystal habits including higher purity, SPAN in a range of 1-2 and an aspect ratio of 1:(1-2) cannot be obtained by direct crystal precipitation at a water temperature above 40° C.

### Example 3

Ropivacaine or ropivacaine hydrochloride was recrystallized by different refining methods to obtain a recrystallized product, which was further prepared into a ropivacaine suspension with a particle size of 8-10 μm; and the effect of the crystal habits on the properties of a preparation was investigated. The preparation method was as follows.
1. Solvents were removed from ropivacaine crystals prepared in Comparative examples 1-3 and Example 1, and ropivacaine crystals prepared in Example 2 by performing incubation at 90° C. for 90 min, by means of suction filtration to obtain relatively dry solids.
2. 0.05 g of polysorbate 80, 0.5 g of sodium carboxymethylcellulose and 2.0 g of mannitol were weighed, and dissolved with 80 g of water to obtain an accessory material solution.
3. The accessory material solution in step 2 was mixed with 2 g of refined ropivacaine, and the weight was fixed to 100 g, followed by shear dispersion at a high speed for 20 min with a Fluke shear head, so that the solids were dispersed completely and uniformly.
4. A particle size variable was controlled: the suspensions in Comparative examples 1-3 were homogenized, and the particle size was controlled to be 8-10 μm.
5. The suspensions were aliquoted in vials, freeze-dried, and stored.
6. The length-to-width ratios, D₁₀, D₅₀, D₉₀ and particle size distribution width (SPAN) values of ropivacaine in Compositions 1-5 were determined, respectively. The test methods were the same as above, and the results were shown in Table 4.

The freeze-dried finished preparations were taken out, and the particle size of each redissolved preparation in a redissolved state was detected. The results were shown in Table 4:

TABLE 4
Detection of freeze-dried preparations in redissolved state

| | | Composition Serial Nos. | | | | |
|---|---|---|---|---|---|---|
| | | 1 | 2 | 3 | 4 | 5 |
| Refining method of | | Comparative | Comparative | Comparative | Example 1 | Example 2 |
| bulk drug | | example 1 | example 2 | example 3 | | |
| Aspect ratio | | 1:(4-10) | >1:10 | Irregular | 1:(1-2) | 1:(1-2) |
| Particle | D₁₀/μm | 2.2 | 2.00 | 2.19 | 0.90 | 4.04 |
| size | D₅₀/μm | 9.23 | 9.35 | 9.24 | 2.48 | 8.13 |
| | D₉₀/μm | 17.86 | 35.59 | 22.64 | 5.24 | 14.6 |

| | | | | | |
|---|---|---|---|---|---|
| Particle size | 1.7 | 3.6 | 2.2 | 1.8 | 1.3 |
| distribution width | | | | | |
| (SPAN) | | | | | |

As can be seen from Table 4, among the suspensions with D₅₀ of 8-10 μm obtained by a homogenization process in Comparative examples 1-3 and the suspension obtained by directly mixing ropivacaine with accessory materials in Example 2, the suspension composition 5 prepared from the ropivacaine crystal in Example 2 of the present disclosure had the narrowest particle size distribution and the lowest SPAN value.

### Example 4: Comparison of Specific Surface Areas of Ropivacaine

Using recognized technologies based on Brunauer, Emmett and Teller theories, the specific surface areas of ropivacaine having different crystal habits in Comparative examples 1-3 and ropivacaine obtained by incubation at 90° C. for 90 min in Example 2 were measured by physical adsorption of nitrogen on the surfaces of samples in each batch.

Crystal surface energy was detected by inverse gas chromatography (IGC). A sample of approximately 300-600 mg was loaded into a separate iGC silanized glass column, followed by operations under surface coverage with a series of alkane and polar probe molecules, so as to determine the dispersed surface energy (SD) and acid-base adsorption free energy (AGSP). In this study, a sample column was pretreated with 10 ml/min of nitrogen carrier gas for 1 h at 30° C. and relative humidity of 0%. The experiment was performed at 30° C. with a total nitrogen flow rate of 10 ml/min, followed by dead volume correction using methane.

The specific surface areas of all samples were determined by a gas adsorption method. The results were shown in Table 5.

TABLE 5
Specific surface areas determined from octane adsorption
isotherm used for peak maximum retention time

| | | Monolayer | | BET specific |
|---|---|---|---|---|
| Sample | Absorption | capacity | R² (correlation | surface area |
| information | coefficient | mMol/g | coefficient) | m²/g |
| Comparative | 1.8108 | 0.0018 | 0.9965 | 0.7171 |
| example 1 | | | | |
| Comparative | 1.5576 | 0.0061 | 0.9939 | 2.3830 |
| example 2 | | | | |
| Comparative | 1.4972 | 0.002 | 0.9955 | 0.7638 |
| example 3 | | | | |
| Example 2 | 1.8428 | 0.0015 | 0.9955 | 0.5904 |

The results showed that the ropivacaine crystal prepared in Example 2 had the smallest specific surface area and more uniform surface energy, and thus had a more stable flow behavior.

### Example 5: Comparison of In-Vitro Release Experiments

The in-vitro release rates of Compositions 1-3 and Composition 5 prepared in Example 3 were tested, and blended to 20 mg/ml with a CZIS dissolution medium (i.e., a 0.05 M disodium hydrogen phosphate solution (pH 9.0)). Redissolved medicine liquor was extracted with a 1 ml syringe, and the corresponding dissolution method was operated. A paddle method loading slot was pushed manually to shake the medicine liquor (containing approximately 18 mg of ropivacaine) in the 1 ml syringe well; a needle was pulled out to allow the syringe to be placed on a paddle method loading port perpendicular to the horizontal plane; and after all the medicine liquor was injected into a dissolution cup, a threaded syringe filter was installed on a sampling port, and the solution at each time point was filtered on line.

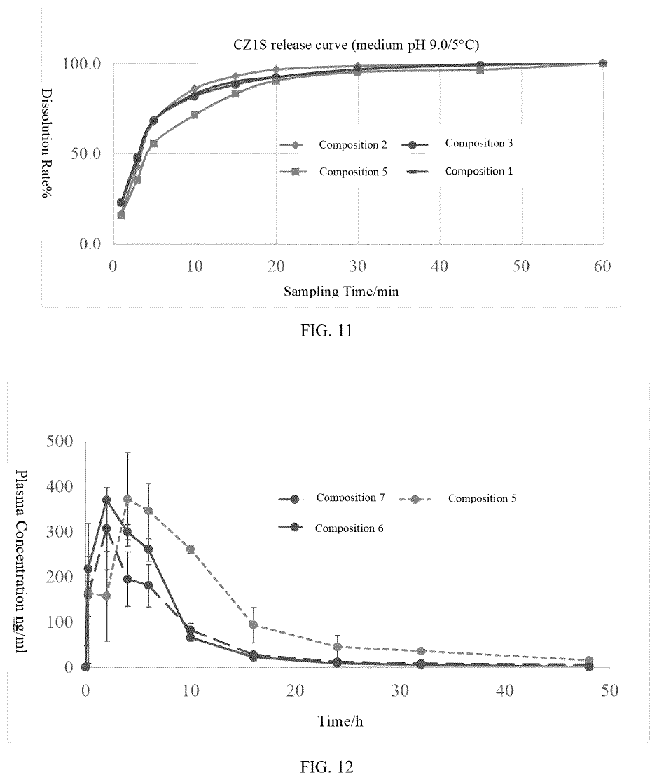

The results were shown in FIG. 11 indicated that the release rates were: Composition 2>Composition 1> Composition 3>Composition 5. That is, a preparation containing the ropivacaine crystal having a specific morphology, specific length-to-width ratio and specific particle size distribution width (SPAN) value prepared in the present disclosure had the lowest release rate and the strongest sustained-release ability, and thus was very valuable for clinical applications.

### Example 6: In-Vivo Studies

In this example, the effects of the particle size of the crystal on Pk was studied.

A suspension of Composition 1 was further homogenized once at a homogenization pressure of 1500 bar to obtain a suspension of Composition 6 with D₅₀=4.66 μm and SPAN=1.52. A suspension of Composition 2 was further homogenized five times to obtain a suspension of Composition 7 with D₅₀=2.09 μm and SPAN=1.36.

Male SD rats were administered intramuscularly at a dose of 31.5 mg/kg, and plasma at different time points was collected and detected by an LC-MS/MS method to investigate pharmacokinetic characteristics of each prescription under a set administration form. The detected plasma concentration-time curve was shown in the drawing, and the detected pharmacokinetic parameters were shown in Table 6 and FIG. 12.

TABLE 6
Pharmacokinetic parameters of SD rats after
intramuscular injection of CZ1S control group

| | | | Compo- | Compo- | Compo- |
|---|---|---|---|---|---|
| | Pharmacokinetic parameters | | sition 5 | sition 6 | sition 7 |
| | Particle size | D₁₀/μm | 4.04 | 1.11 | 1.23 |
| | | D₅₀/μm | 8.13 | 2.09 | 4.66 |
| | | D₉₀ μm | 14.6 | 3.96 | 8.32 |
| SPAN | | | 1.3 | 1.4 | 1.5 |
| | t_{1/2} | h | 11.49 | 4.49 | 4.62 |
| | Tₘₐₓ | h | 2.67 | 2.00 | 2.00 |
| | Cₘₐₓ | ng/mL | 402.85 | 369.03 | 306.1 |
| | AUC₍₀₋ₜ₎ | h*ng/ml | 5352.50 | 2817.4 | 2429.52 |
| | AUC_{(0-∞)} | h*ng/ml | 5637.25 | 2857.96 | 2468.55 |

The results indicated that, through mechanical means such as homogenization and grinding, the ropivacaine crystals having different crystal habits were granulated into relatively regular shapes, and the SPAN value of the crystals was ≤1.5. Although the SPAN value of the crystals could be reduced, the particle sizes of the crystals decreased at the same time, and the sustained-release ability of the preparation was not good.

### Example 7: Comparison of Pharmacokinetic Parameters of Different Crystal Habits

The dynamic change characteristics of SD rats after a single subcutaneous or sciatic plexus injection of Composition 1, Composition 2, Composition 3 and Composition 5 in vivo were studied to obtain relevant pharmacokinetic parameters.

Method: twenty-four male SD rats were randomly divided into eight groups, with three rats in each group, which were subcutaneous injection (120 mg/kg) groups and sciatic plexus injection (80 mg/kg) dose groups, both of which were administered as a single dose with a volume of 2 mL/kg. Blood samples were collected before and at 0.5, 1, 2, 4, 8, 12, 24, 48 and 72 h after administration in each dose group. The concentration of ropivacaine in each plasma sample was detected by an LC-MS/MS method. The relevant pharmacokinetic parameters were detailed in Table 7 and Table 8.

TABLE 7
Main pharmacokinetic parameters of ropivacaine in plasma
after single subcutaneous injection in SD rats

| | AUCₗₐₛₜ | AUC_{0-∞} | | | |
|---|---|---|---|---|---|
| | (h · | (h · | Cₘₐₓ | Tₘₐₓ | t_{1/2} |
| Groups | μg/mL) | μg/mL) | (μg/mL) | (h) | (h) |
| Composition 1 | 24.1 ± | 24.8 ± | 2.30 ± | 3.0 ± | 13 ± |
| | 5.55 | 5.27 | 1.76 | 1.7 | 4.4 |
| Composition 2 | 21.5 ± | 21.6 ± | 0.950 ± | 2.0 ± | 8.7 ± |
| | 4.50 | 4.72 | 0.546 | 1.6 | 3.2 |
| Composition 3 | 19.7 ± | 20.0 ± | 1.22 ± | 1.2 ± | 12 ± |
| | 5.94 | 5.86 | 0.821 | 0.76 | 3.8 |
| Composition 5 | 24.4 ± | 25.3 ± | 1.37 ± | 2.7 ± | 16 ± |
| | 4.01 | 2.07 | 0.366 | 1.2 | 2.1 |

TABLE 8
Main pharmacokinetic parameters of ropivacaine in plasma
after single sciatic plexus injection in SD rats

| | AUCₗₐₛₜ | AUC_{0-∞} | | | |
|---|---|---|---|---|---|
| | (h · | (h · | Cₘₐₓ | Tₘₐₓ | t_{1/2} |
| Groups | μg/mL) | μg/mL) | (μg/mL) | (h) | (h) |
| Composition 1 | 11.2 ± | 11.5 ± | 0.619 ± | 2.5 ± | 10 ± |
| | 0.179 | 0.104 | 0.0403 | 1.2 | 2.0 |
| Composition 2 | 10.6 ± | 10.8 ± | 0.595 ± | 2.0 ± | 9.3 ± |
| | 0.147 | 0.192 | 0.199 | 0.0 | 0.69 |
| Composition 3 | 11.8 ± | 12.0 ± | 0.646 ± | 2.0 ± | 8.6 ± |
| | 0.237 | 0.218 | 0.185 | 0.82 | 2.5 |
| Composition 5 | 11.1 ± | 11.6 ± | 0.656 ± | 3.0 ± | 11 ± |
| | 1.33 | 1.47 | 0.110 | 1.2 | 2.8 |

The results indicated that suspension injections having crystal habits in the present disclosure had more excellent pharmacokinetic parameters in vivo: Composition 2 and Composition 3 had smaller half-life periods, and shorter time to peak, which may be due to high proportions and fast release rates of crystals with smaller particle sizes in the compositions; and Composition 5 had a longer half-life period, indicating that the crystal habits protected by this patent could be more prone to achieving sustained release.

What is claimed is:

1. A ropivacaine crystal, wherein the ropivacaine crystal has an aspect ratio of 1:(1-2).

2. The ropivacaine crystal according to claim 1, wherein the ropivacaine crystal has an aspect ratio of 1:(1-1.9), or 1:(1-1.8), or 1:(1-1.7), or 1:(1-1.6), or 1:(1-1.5), or 1:(1-1.4), or 1:(1-1.3), or 1:(1-1.2), or 1:(1-1.1), or 1:1.

3. The ropivacaine crystal according to claim 1, wherein at least 80%, at least 85%, at least 90%, or at least 95% of the ropivacaine crystals have the aspect ratio of 1:(1-2).

4. The ropivacaine crystal according to claim 1, wherein D₅₀ of the ropivacaine crystal at room temperature is 5-10 μm.

5. The ropivacaine crystal according to claim 4, wherein the D₅₀ of the ropivacaine crystal at the room temperature is 5.0 μm, 5.1 μm, 5.2 μm, 5.3 μm, 5.4 μm, 5.5 μm, 5.6 μm, 5.7 μm, 5.8 μm, 5.9 μm, 6.0μ, 6.1μ, 6.2 μm, 6.3 μm, 6.4 μm, 6.5 μm, 6.6 μm, 6.7 μm, 6.8μ, 6.9 μm, 7.0 m, 7.1 μm, 7.2 μm, 7.3 μm, 7.4μ, 7.5μ, 7.6μ, 7.7μ, 7.8μ, 7.9μ, 8.0μ, 8.1μ, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, 9.9 μm, or 10.0 μm.

6. The ropivacaine crystal according to claim 1, wherein D₅₀ of at least 80%, at least 85%, at least 90%, or at least 95% of the ropivacaine crystals at room temperature is 5-10 μm.

7. A ropivacaine crystal, wherein the ropivacaine crystal has a particle size distribution width value of less than or equal to 2.

8. The ropivacaine crystal according to claim 7, wherein the ropivacaine crystal has a particle size distribution width value of 1-2.

9. The ropivacaine crystal according to claim 7, wherein the ropivacaine crystal has a particle size distribution width value of 1, 1.1, 1.2, 1.3, 1.4, 1.5, 1.6, 1.7, 1.8, 1.9, and 2.0.

10. The ropivacaine crystal according to claim 1, wherein the ropivacaine crystal has the aspect ratio of 1:(1-2) and a particle size distribution width value of less than or equal to 2.

11. The ropivacaine crystal according to claim 1, wherein the ropivacaine crystal has a particle morphology shown in FIG. 5.

12. The ropivacaine crystal according to claim 1, wherein the ropivacaine crystal has an XRPD diffraction diagram shown in FIG. 1.

13. A method for preparing a ropivacaine crystal, comprising:
dissolving ropivacaine hydrochloride in a solvent, and stirring to dissolve;
after complete dissolution, adjusting a pH to alkalinity and precipitating crystals to obtain a solution; and
heating the solution, and performing incubation, wherein
the solvent is water.

14. The method according to claim 13, wherein a temperature of a crystal precipitation is less than 40° C.

15. The method according to claim 13, wherein a temperature of a crystal precipitation is room temperature.

16. The method according to claim 13, wherein a pH value is adjusted to be greater than 7.

17. The method according to claim 13, wherein the solution is heated to 80-100° C.

18. The method according to claim 13, wherein when an incubation temperature is greater than or equal to 90° C., an incubation time is more than or equal to 10 min.

19. A pharmaceutical composition, comprising the ropivacaine crystal according to claim 1.

20. A method for a treatment or prevention of postoperative pain, comprising administering to a subject in need thereof the ropivacaine crystal according to claim 1.
